(19) United States
(12) Patent Application Publication
(10) Pub. No.: US 2006/0100642 A1
(43) Pub. Date: May 11, 2006

(54) Control of robotic manipulation

(76) Inventors: Guang-Zhong Yang, Epsom, Surrey (GB); Ara Darzi, Gerrards Cross Buckinghamshire (GB)

Correspondence Address: HICKMAN PALERMO TRUONG & BECKER, LLP, 2055 GATEWAY PLACE, SUITE 550, SAN JOSE, CA, 95110, US

(21) Appl. No.: 10/529,023

(22) PCT Filed: Sep. 25, 2003
(86) PCT No.: PCT/GB03/04077

§ 371 Date: Dec. 22, 2005

(30) Foreign Application Priority Data
Sep. 25, 2002 (GB) 0222265.1

(51) Int. Cl. A61B 19/00

(52) U.S. Cl. 606/130

(57) ABSTRACT

In a remote controlled robotic manipulator 20 a motion sensor 26 senses motion of a region of an object to be manipulated. A controller 50 locks motion of the robotic manipulator 26 relative to the region of the object and also selects the region of the object to be sensed. As a result the frame of reference of the manipulator is locked to the relevant region of the object to be manipulated improving ease of control and manipulation.

### CROSS-REFERENCE TO RELATED APPLICATIONS; PRIORITY CLAIM

This application is a submission under 35 U.S.C. §371 based on prior international application PCT/GB2003/004077, filed 25 Sep. 2003, which claims priority from United Kingdom application 0222265.1, filed 25 Sep. 2002, entitled “Control of Robotic Motion,” the entire contents of which are hereby incorporated by reference as if fully set forth herein.

### COPYRIGHT NOTICE

A portion of the disclosure of this patent document contains material which is subject to copyright protection. The copyright owner has no objection to the facsimile reproduction of the patent disclosure, as it appears in the Patent & Trademark Office patent file or records, but otherwise reserves all copyright rights whatsoever.

### FIELD OF THE INVENTION

The invention relates to control of robotic manipulation; in particular motion compensation in robotic manipulation. The invention further relates to the use of stereo images.

### BACKGROUND

Robotic manipulation is known in a range of fields. Typical systems include a robotic manipulator such as a robotic arm which is remote controlled by a user. For example the robotic arm may be configured to mirror the actions of the human hand. In that case a human controller may have sensors monitoring actions of the controller's hand. Those sensors provide signals allowing the robotic arm to be controlled in the same manner. Robotic manipulation is useful in a range of applications, for example in confined or in miniaturized/microscopic applications.

One known application of robotic manipulation is in medical procedures such as surgery. In robotic surgery a robotic arm carries a medical instrument. A camera is mounted on or close to the arm and the arm is controlled remotely by a medical practitioner who can view the operation via the camera. As a result keyhole surgery and microsurgery can be achieved with great precision. A problem found particularly in medical procedures but also in other applications arises when it is required to operate on a moving object or moving surface such as a beating heart. One known solution in medical procedures is to hold the relevant surface stationary. In the case of heart surgery it is known to stop the heart altogether and rely on other life support means while the operation is taking place. Alternatively the surface can be stabilized by using additional members to hold it stationary. Both techniques are complex, difficult and increase the stress on the patient.

One proposed solution is set out in U.S. Pat. No. 5,971,976 in which a position controller is also included. The medical instrument is mounted on a robotic arm and remotely controlled by a surgeon. The surface of the heart to be operated on is mechanically stabilized and the stabilizer also includes inertia or other position/movement sensors to detect any residual movement of the surface. A motion controller controls the robotic arm or instrument to track the residual movement of the surface such that the distance between them remains constant and the surgeon effectively operates on a stationary surface. A problem with this system is that the arm and instrument are motion locked to a specific point or zone on the heart defined by the mechanical stabilizer but there is no way of locking it to other areas. As a result if the surgeon needs to operate on another region of the surface then the residual motion will no longer be compensated and can indeed be enhanced if the arm is tracking another region of the surface, bearing in mind the complex surface movement of the heart.

The invention is set out in the appended claims. Because the motion sensor can sense motion of a range of points, the controller can determine the part of the object to be tracked. Eye tracking relative to a stereo image allows the depth of a fixation point to be determined.

### BRIEF DESCRIPTION OF DRAWINGS

Embodiments of the invention will now be described, by way of example, with reference to the drawings of which:

FIG. 1 is a schematic view of a known robotic manipulator;

FIG. 2 shows the components of an eye tracking system;

FIG. 3 shows a robotic manipulator according to the invention;

FIG. 4 shows a schematic view of a stereo image display; and

FIG. 5 shows the use of stereo image in depth determination.

### DETAILED DESCRIPTION

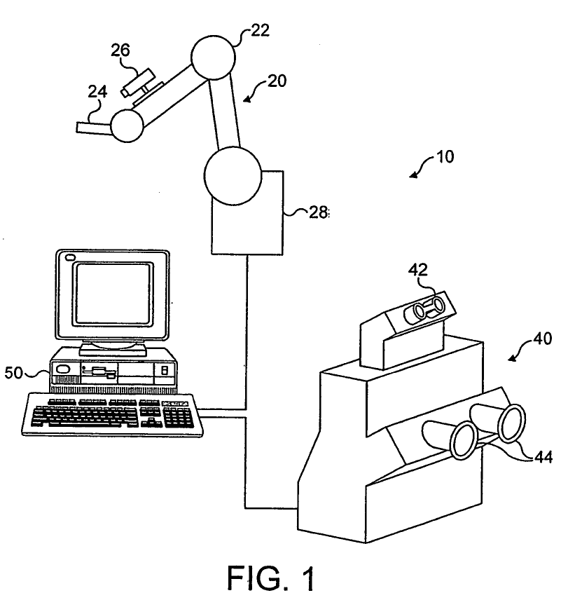

Referring to FIG. 1 a typical arrangement for performing robotic surgery is shown designated generally 10. A robotic manipulator 20 includes an articulated arm 22 carrying a medical instrument 24 as well as the cameras 26. The arm is mounted on a controller 28. A surgical station designated generally 40 includes binocular vision eye pieces 42 through which the surgeon can view a stereo image generated by cameras 26 and control gauntlets 44. The surgeon inserts his hands into the control gauntlets and controls a remote analogue of the robotic manipulator 20 based on the visual feedback from eyepiece 42. Interface between the robotic manipulator 20 and surgical station 40 is via an appropriate computer processor 50 which can be of any appropriate type for example a PC or laptop. The processor 50 conveys the images from camera 26 to the surgical station 40 and returns control signals from the robotic arm analogue controlled by the surgeon via gauntlets 44. As a result a fully fed back surgical system is provided. Such a system is available under the trademark Da Vinci Surgical Systems from Intuitive Surgical, Inc of Sunnyvale Calif. USA or Zeus Robotic Surgical Systems from Computer Motion, Inc Goleta Calif. USA. In use the surgical instrument operates on the patient and the only incision required is sufficient to allow camera vision and movement of the instrument itself as a result of which minimal stress to the patient is introduced. Furthermore using appropriate magnifications/reduction techniques, micro surgery can very easily take place.

As discussed above, it is known to add motion compensation to a system such as this whereby motion sensors on the surface send a movement signal which is tracked by the robotic arm such that the surface and arm are stationary relative to one another. In overview the present invention further incorporates an eye tracking capability at the surgical station 40 identifying which part of the surface the surgeon is fixating on and ensuring that the robotic arm tracks that particular point, the motion of which may vary relative to other points because of the complex motion of the heart's surface. As a result the invention achieves dynamic reference frame locking.

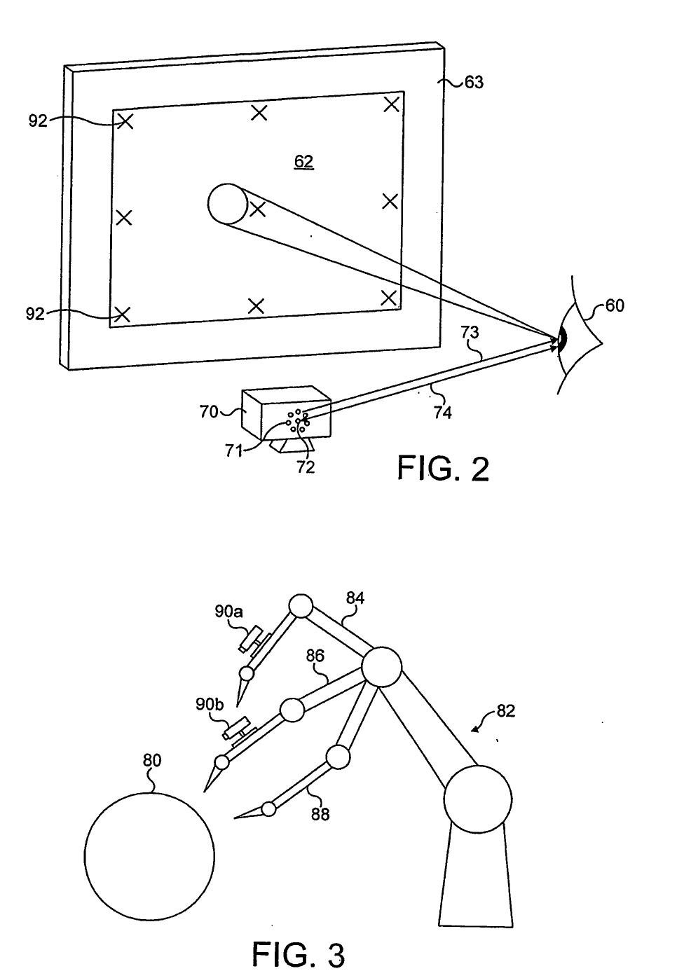

Referring to FIG. 2 an appropriate eye tracking arrangement is shown schematically. The user 60 views an image 62 on a display 63. An eye-tracking device 70 includes one or more light projectors 71 and a light detector 72. In practice the light projectors may be infrared (IR) LEDs and the detector may be an IR camera. The LEDs project light 73 onto the eye of the user 60 and the angle of gaze of the eye can be derived using known techniques by detecting the light 74 reflected onto the camera. Any appropriate eye tracking system may in practice be used for example an ASL model 504 remote eye-tracking system (Applied Science Laboratories, Mass., USA). This embodiment may be particularly applicable when a single camera is provided on the articulated arm 22 of a robotic manipulator and thus a single image is presented to the user. The gaze of the user is used to determine the fixation point of the user on the image 62. It will be appreciated that a calibration stage may be incorporated on initialization of any eye-tracking system to accommodate differences between users' eyes or vision. The nature of any such calibration stage will be well known to the skilled reader.

Referring now to FIG. 3, the robotic arm and tracking system are shown in more detail.

An object 80 is operated on by a robotic manipulator designated generally 82. The manipulator 82 includes 3 robotic arms 84, 86, 88 articulated in any appropriate manner and carrying appropriate operating instruments. Arm 84 and arm 86 each support a camera 90a, 90b displaced from one another sufficient to provide stereo imaging according to known techniques. Since the relative positions of the three arms are known, the position of the cameras in 3D space is also known.

In use the system allows motion compensation to be directed to the point on which the surgeon is fixating (i.e. the point he is looking at, at a given moment). Identifying the fixation point can be achieved using known techniques which will generally be built in with an appropriate eye tracking device provided, for example, in the product discussed above. In the preferred embodiment the cameras are used to detect the motion of the fixation point and send the information back to the processor for control of the motion of the robotic arm.

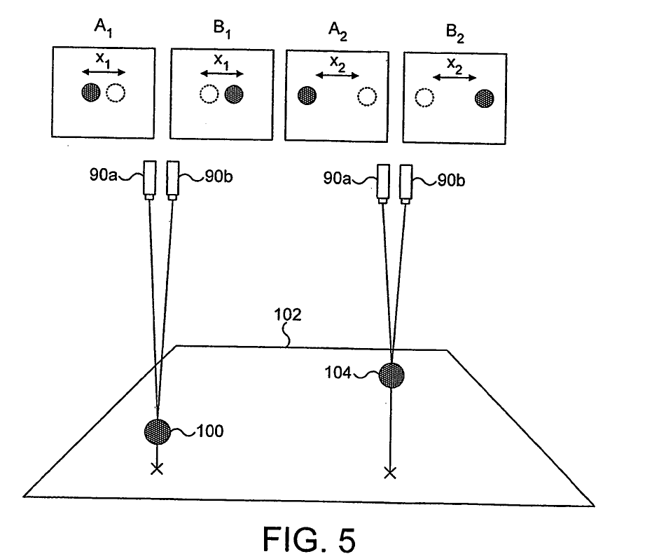

In particular, once, at any one moment, the fixation point position is identified on the image viewed by the human operator, given that the position of the stereo cameras 90a and 90b are known the position of the point on the object 80 can be identified. Alternatively, by determining the respective direction of gaze of each eye, this can be replicated at the stereo camera to focus on the relevant point. The motion of that point is then determined by stereo vision. In particular, referring to FIG. 5 it will be seen that the position of a point can be determined by measuring the disparity in the view taken by each camera 90a, 90b. For example for a relatively distant object 100 on a plane 102 the cameras take respective images A1, B1 defining a distance X1. A more distant object 104 creates images A2, B2 in which the distance between the objects as shown in the respective images is X2. There is an inverse relationship between the distance and the depth of the point. As a result the relative position of the point to the camera can be determined.

In particular, the computer 50 calculates the position in the image plane of the co-ordinates in the real world (so-called “world coordinates”). This may be done as follows:

A 3D point M=[x,y,z]^{T} is projected to a 2D image point m=[x,y,]^{T} through a 3×4 projection matrix P, such that S m=P M, where S is a non-zero scale factor and m=[x, y, 1]^{t} and M=[x,y,z,1]^{t}. In binocular stereo systems, each physical point M in 3D space is projected to m₁ and m₂ in the two image planes, i.e;
S₁m₁=P₁M
S₂m₂=P₂M (1)

If we assume that the world coordinate system is associated with the first camera, we have
P₁=[A|]
P₂=[A′R|A′t] (2)
Where R and t represent the 3×3 rotation matrix and the 3×1 translation vector defining the rigid displacement between the two cameras.

The matrices A and A¹ are the 3×3 intrinsic parameter matrices of the two cameras. In general, when the two cameras have the same parameter settings and with square pixels (aspect ration=1), and the angle (θ) between the two image coordinate axes being π/2 we have:

$$A = [ \begin{matrix}f & 0 & {u}_{0} \\ 0 & f & {v}_{0} \\ 0 & 0 & 1\end{matrix} ] \qquad ( 3 )$$

Where (u₀, v₀) are the coordinates of the image principal point, i.e, the point where points located at infinity in world coordinates are projected.

Generally, matrix A can have the form of

$$A = [ \begin{matrix}{f}_{u} & {f}_{u} cot θ & {u}_{0} \\ 0 & {f}_{v} / sin θ & {v}_{0} \\ 0 & 0 & 1\end{matrix} ] \qquad ( 4 )$$

Where fᵤ and fᵥ correspond to the focal distance in pixels along the axes of the image. All parameters of A can be computed through classical calibration method (e.g. as described in the book by O. Faugeras, “Three-Dimensional Computer Vision: a Geometric Viewpoint”, MIT press, Cambridge, Mass., 1993).

Known techniques for determining the depth are for example as follows. Firstly, the apparatus is calibrated for a given user. The user looks at predetermined points on a displayed image and the eye tracking device tracks the eye(s) of the user as they look at each predetermined point. This sets the user's gaze within a reference frame (generally two-dimensional if one image is displayed and three-dimensional if stereo images are displayed). In use, the user's gaze on the image(s) is tracked and thus the gaze of the user within this reference frame is determined. The robotic arms 84, 86 then move the cameras 90a, 90b to focus on the determined fixation point.

For instance, consider FIG. 2 again which shows a user 60, an image 62 on a display 63 and an eye-tracking device 70. In use, the tracking device 70 is first calibrated for the user. This involves the computer 50 displaying on the display a number of pre-determined calibration points, indicated by 92. A user is instructed to focus on each of these in turn (for instance, the computer 50 may cause each calibration point to be displayed in turn). As the user stares at a calibration point, the eye-tracking device 70 tracks the gaze of the user. The computer then correlates the position of the calibration point with the position of the user's eye. Once all the calibration points have been displayed to a user and the corresponding eye position recorded, the system has been calibrated to the user.

Subsequently a user's gaze can be correlated to the part of the image being looked at by the user. For each eye, the coordinates [x₁, y₁] and [xᵣ, yᵣ] are known from each eye tracker from which [x, y, z]^{T} can be calculated from Equations (1)-(4).

By carrying out this step across time the motion of the point fixated on by the human operator can be tracked and the camera and arm moved by any appropriate means to maintain a constant distance from the fixation point. This can either be done by monitoring the absolute position of the two points and keeping it constant or by some form of feedback control such as using PID control. Once again the relevant techniques will be well known to the skilled person.

It will be further recognized that the cameras can be focused or directed towards the fixation point determined by eye tracking, simply by providing appropriate direction means on or in relation to the robotic arm. As a result the tracked point can be moved to centre screen if desired.

In the preferred embodiment the surgical station provides a stereo image via binocular eyepiece 42 to the surgeon, where the required offset left and right images are provided by the respective cameras mounted on the robotic arm.

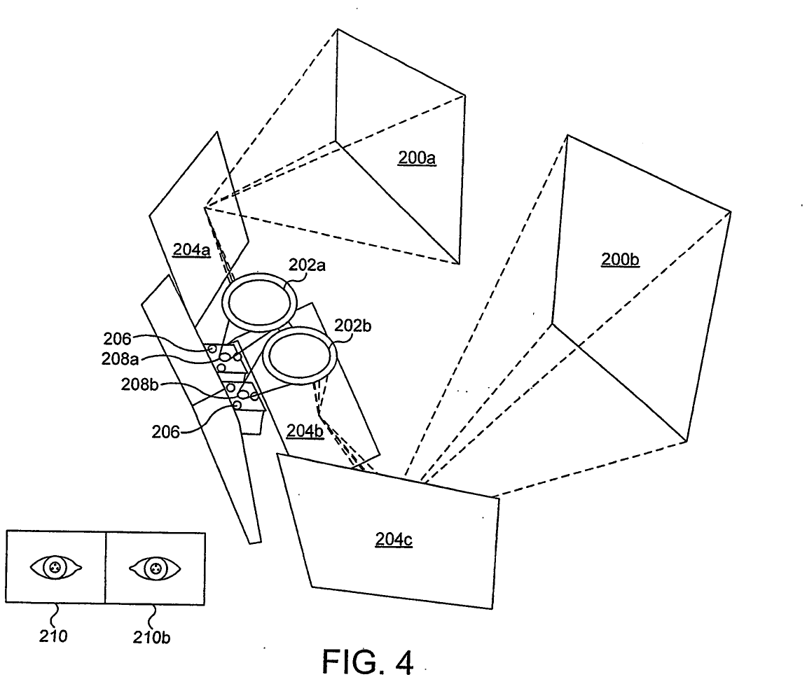

According to a further aspect of the invention enhanced eye tracking in relation to stereo images is provided. Referring to FIG. 4, a further embodiment of the invention is shown. The system requires left and right images slightly offset to provide, when appropriately combined, a stereo image as well known to the skilled reader. Images of a subject being viewed are displayed on displays 200a, 200b. These displays are typically LCD displays. A user views the images on the displays 200a, 200b through individual eyepieces 202a, 202b via intermediate optics including mirrors 204a, b, c (and any appropriate lens although any appropriate optics can of course be used).

Eye tracking devices are provided for each individual eyepiece. The eye-tracking device includes light projectors 206 and light detectors 208. In a preferred implementation, the light projectors are IR LEDs and the light detector comprises an IR camera for each eye. An IR filter may be provided in front of the IR camera. The images (indicated in FIG. 4 by the numerals 210a, 210b) captured by the light detectors 208a, 208b show the position of the pupils of each eye of the user and also the Purkinje Reflections of the light sources 206.

The angle of gaze of the eye can be derived using known techniques by the detecting the reflected light.

In a preferred, known implementation Purkinje images are formed by light reflected from surfaces in the eye. The first reflection takes place at the anterior surface of the cornea while the fourth occurs at the posterior surface of the lens of the eye. Both the first and fourth Purkinje images lie in approximately the same plane in the pupil of the eye and, since eye rotation alters the angle of the IR beam from the IR projectors 206 with respect to the optical axis of the eye, and eye translations move both images by the same amount, eye movement can be obtained from the spatial position and distance between the two Purkinje reflections. This technique is commonly known as the Dual-Purkinje Image (DPI) technique. DPI also allows for the calculation of a user's accommodation of focus i.e. how far away the user is looking. Another eye tracking technique subtracts the Purkinje reflections from the nasal side of the pupil and the temporal side of the pupil and uses the difference to determine the eye position signal. Any appropriate eye tracking system may in practice be used for example an ASL model 504 remote eye-tracking system (Applied Science Laboratories, MA, USA).

By tracking the individual motion of each eye and identifying the fixation point F on the left and right images 200a, 200b, not only the position of the fixation point in the X Y plane (the plane of the images) can be identified but also the depth into the image, in the Z direction.

Once the eye position signal is determined, the computer 50 uses this signal to determine where, in the reference field, the user is looking and calculates the corresponding position on the subject being viewed. Once this position is determined, the computer signals the robotic manipulator 82 to move the arms 84 and/or 86 which support the cameras 90a and 90b to focus on the part of the subject determined from the eye-tracking device, allowing the motion sensor to track movement of that part and hence lock the frame of reference to it.

Although the invention has been described with reference to eye tracking devices that use reflected light, other forms of eye tracking may be used, e.g. measuring the electric potential of the skin around the eye(s) or applying a special contact lens and tracking its position.

It will be appreciated that the embodiments above and elements thereof can be combined or interchanged as appropriate. Although specific discussion is made of the application of the invention to surgery, it will be recognized that the invention can be equally applied in many other areas where robotic manipulation or stereo imaging is required. Although stereo vision is described, monocular vision can also be applied. Also other appropriate means of motion sensing can be adopted, for instance, by the use of casting structured light onto the object and observing changes as the object moves, or by using laser range finding. These examples are not supposed to be limiting.

1. A remote controlled robotic manipulator for manipulating a moving object comprising a motion sensor for sensing motion of a region of an object to be manipulated, and a controller for locking motion of the robotic manipulator relative to the region of the object based on the sensed motion, wherein the controller further controls for which region of the object the motion sensor senses motion.

2. A manipulator as claimed in claim 1 in which the motion sensor is controllable by a human user.

3. A manipulator as claimed in claim 2 in which the motion sensor is controllable by tracking a visual fixation point of the user.

4. A manipulator as claimed in claim 3 in which the user views a remote representation of the object.

5. A method of identifying a visual fixation point of a user observing a stereo image formed by visually superposing mono images comprising the steps of presenting one mono image to each user eye to form the stereo image and tracking the fixation point of each eye.

6. A method as claimed in claim 5 in which the three dimensional position of the visual fixation point is determined.

7. An apparatus for identifying a fixation point in a stereo image comprising first and second displays for displaying mono images, a stereo image presentation module for visually super-posing the mono images to form the stereo image and an eye tracker for tracking a fixation point of each eye.

8. A manipulator as claimed in claim 1, wherein the region is within a human undergoing surgery and wherein the object is a tissue that is the subject of the surgery.

9. A manipulator as claimed in claim 1, wherein the controller determines the region of the object based on a signal from an eye tracking apparatus that tracks a visual fixation point of one or more eyes of a user.

10. A manipulator as claimed in claim 9, wherein the eye tracking apparatus identifies the visual fixation point of the user who is observing a stereo image formed by visually superposing mono images, comprising the steps of presenting one mono image to each user eye to form the stereo image and tracking the fixation point of each eye.

11. A manipulator as claimed in claim 10 in which a three-dimensional position of the visual fixation point is determined.

12. A manipulator as claimed in claim 10, further comprising left and right LCD displays that display left and right images.

13. A method as claimed in claim 5, wherein the mono images are obtained from sensors that are observing a human body as part of a surgery.

14. An apparatus as recited in claim 7, wherein the eye tracker determines a three-dimensional position of the fixation point.

15. An apparatus as recited in claim 7, further comprising a remote controlled robotic manipulator for manipulating a moving object, a motion sensor for sensing motion of a region of an object to be manipulated, and a controller for locking motion of the robotic manipulator relative to the region of the object based on the sensed motion, wherein the controller further controls for which region of the object the motion sensor senses motion.

16. An apparatus as claimed in claim 15 in which the motion sensor is controllable by a human user.

17. An apparatus as claimed in claim 16, wherein the eye tracker determines a three-dimensional position of the fixation point, and wherein the eye tracker controls the motion sensor.

18. An apparatus as claimed in claim 7 in which a user views a remote representation of the object.

19. An apparatus as claimed in claim 15, wherein the region is within a human undergoing surgery and wherein the object is an organ that is the subject of the surgery.

20. An apparatus as claimed in claim 7, wherein each of the mono images depicts a region within a human undergoing surgery, and wherein the eye tracker tracks the fixation point of each eye of a surgeon.
